(19) United States
(12) Patent Application Publication
(10) Pub. No.: US 2012/0101359 A1
(43) Pub. Date: Apr. 26, 2012

(54) FIXATION USING MULTIPLE CUSIONS OF TWO DIFFERENT TYPES

(75) Inventors: Roland Eichardt, Nerkewitz (DE); Alistair Mcewan, Edgecliff Nsw (AU); Matthias Hamsch, Aachen (DE)

(73) Assignee: KONINKLIJKE PHILIPS ELECTRONICS N.V., Eindhoven (NL)

(21) Appl. No.: 13/146,265

(22) PCT Filed: Jan. 25, 2010
(86) PCT No.: PCT/IB2010/050314

§ 371 Date: Jan. 9, 2012

(30) Foreign Application Priority Data
Jan. 30, 2009 (EP) 09151769.8

(51) Int. Cl. A61B 6/04; A61G 15/12

(52) U.S. Cl. 600/407; 128/845

(57) ABSTRACT

The present invention provides a fixation device comprising at least two inflatable and deflatable cushions (3,4) of two different types. The cushion of the first type (3) is deformable when inflated and not deformable when deflated, while the cushion of the second type (4) is arranged to at least partially expand into the direction of a cushion of the first type upon inflation. The fixation device provides for an improved fit and will easily and perfectly adapt to the shape and size of almost every body part.

### FIELD OF THE INVENTION

The invention relates to the field of medical imaging techniques, in particular to fixation devices used for the acquisition of high quality imaging data.

### BACKGROUND OF THE INVENTION

Various medical imaging techniques such as magnetic resonance imaging (MRI), magnetic induction tomography (MIT), computer tomography (CT) and others are known today. Almost all of these techniques are sensitive to movement artifacts which result from movements of the examined object and lead to disturbed or even unusable images. Therefore, the period of image acquisition of most of such imaging techniques can be divided into several periods of free movement and fixation, wherein image data are only obtained during the state of fixation, wherein the movements of the object are minimized or avoided to allow for successful image reconstructions.

Conventional devices for the fixation of the patient usually include a frame and a fixation structure for fixing the frame to a patient's body part to be examined. However, the application of such fixation devices is laborious and time-consuming as these have to be adapted and attached to the respective body part of the patient. Furthermore, after such a rigid fixation device has been attached to the patient's body the patient often is immobilized for the whole duration of the subsequent examination, which can, for example in case of MIT monitoring, take several hours. Thus, conventional fixation devices restrict the patient's free movement for long periods of time which may be very unpleasant for the patient and/or may lead to claustrophobic feelings during such examinations. Furthermore, particularly the application of fixation devices for the head is prone to error and often results in an imperfect fit of the device, in turn leading to movement artifacts.

### SUMMARY OF THE INVENTION

It is the object of the invention to provide a fixation device with a good fit that can be easily applied and is convenient for the patient, even for examinations of longer duration.

This object is achieved by the fixation device according to claim 1. Advantageous embodiments are given in the dependent claims.

The fixation device according to the invention comprises at least two inflatable and deflatable cushions of two different types, wherein the cushion of the first type is deformable when inflated and not deformable when deflated and the cushion of the second type is arranged to at least partially expand into the direction of a cushion of the first type upon inflation. The fixation device is preferably suitable for the fixation of a body part of a patient, more preferably for the fixation of the head of a patient.

The inflatable and deflatable cushions according to the invention provide for an improved fit and thus for an improved fixation of the body part of the patient to be examined as they will easily adapt to the shape and size of almost every body part. Furthermore, they can be applied and removed quickly, i.e. within seconds. This allows dividing the complete imaging process into sections of fixation wherein no movement of the examined body part is allowed and the images are acquired and sections of non-fixation, wherein movement is allowed and no images are acquired. When the body part is fixed its position can be registered which can be used for later image reconstruction by various methods. Means for the registration and methods for the image reconstruction are known to the skilled person.

The cushions of the first type are deformable or flexible when they are inflated, e.g. filled with air, and will adapt to the shape of the object placed thereon. Thus, if an object is placed onto an inflated cushion of the first type the cushion will change its shape to match the shape of the object. In the deflated state the cushion is stiff and not deformable any longer, keeping its current shape, i.e. the shape it took on when the object was placed onto the inflated cushion.

The cushions of the second type can be expanded by inflation and shrunk by deflation and they are arranged to at least partially expand into the direction of a cushion of the first type. Thus, when inflating, a cushion of the second type will eventually contact the object placed on the cushion of the first type and exert a force against said object. In turn, the object will be pressed into and fixated by the deflated or deflating cushion of the first type. An advantage of the fixation device of the present invention is that it provides for a non-invasive fixation, can easily and quickly be applied and is suitable for different shapes and sizes objects to be fixated.

In one embodiment of the invention the fixation device is a fixation device to be used in MRI, MIT and/or CT applications.

In a further embodiment of the invention the fixation device is suitable for the fixation of an object selected from the group consisting of an extremity, an upper extremity, a lower extremity, a limb, a joint, a leg, an arm, an elbow, a forearm, a wrist, a knee, a hand, the neck and/or the head of a patient. Preferably, the fixation device is suitable for fixing the head of a patient, i.e. the fixation device is a head fixation device.

The casing of the cushions can be made from any suitable material; preferably it is made of a synthetic material, more preferably it is made of plastic. Most preferably, the casing of the cushions is of a semi-rigid material.

In another embodiment of the invention the cushion of the first type comprises a natural and/or synthetic elastic material with air chambers. Upon deflation of the cushion of the first type the air is removed from said air chambers, resulting in a stiffening of the material. In preferred embodiments the elastic material with air chambers is a foam, preferably a polyurethane foam, or Styrofoam. In a further preferred embodiment the cushion of the second type partly surrounds the object placed thereon. For example, the object may not only contact the cushion with its bottom surface but also with one or more lateral surfaces.

In a further embodiment of the invention the cushion of the second type is only filled with air and/or comprises a natural and/or synthetic elastic material with air chambers. In a preferred embodiment the cushion of the second type comprises soft foam, preferably plastic foam. In another preferred embodiment a soft material is attached to the surface of the cushion of the second type. This material will then contact the object placed on the cushion of the first type. In another preferred embodiment the cushion of the second type takes in a predefined shape upon inflation and becomes stiff. Preferably, the predefined shape matches the object to be examined. In a further preferred embodiment of the invention the cushion(s) of the second type are positioned above the cushions of the cushions of the second type if the fixation device is assembled into, for example, an MIT, an MRI or a CT device.

In a further embodiment of the invention ≧2, ≧3, ≧4, ≧5 cushions of the first type and/or ≧2, ≧3, ≧4, ≧5 cushions of the second type are used.

In a preferred embodiment the cushion of the first type is used to fixate and/or the lower part of a head and/or the neck. In a further preferred embodiment the cushion of the second type is used to contact the forehead portion of the head and/or the area of the root of the nose. This provides for the advantage that sensitive areas of the head are not contacted by the cushion of the second type. A further advantage is that this fixation device provides for a non-invasive fixation, can easily and quickly be applied and is suitable for different shapes and sizes of heads.

In a preferred embodiment, two cushions of the first type are used, wherein one is used to fixate the lower part of the head and another one is used to fixate the neck of the patient. In another preferred embodiment two cushions of the second type are used, wherein one is used to contact the forehead portion of the head and the other one is used to contact the area of the root of the nose. This has the advantage that an especially good fit can be achieved, wherein no extremely sensitive areas of the head need to be contacted.

In another embodiment the fixation device comprises at least one vacuum pump and/or air pump. In another preferred embodiment each cushion of the fixation device is connected to a separate vacuum pump and/or air pump. In a preferred embodiment the cushion(s) of the first type and the cushion(s) of the second type are connected to at least one vacuum pump and/or air pump that independently provide for the inflation and deflation of each cushion. In a further embodiment the fixation device of the present invention thus comprises at least one vacuum pump and/or air pump arranged as described above.

In another embodiment the fixation device comprises at least one valve that supports the inflation and/or deflation process. Preferably, each line connecting a cushion to a vacuum pump and/or air pump comprises a valve. In a further embodiment the fixation device of the present invention thus comprises at least one valve arranged as described above. In another embodiment the fixation device comprises at least one means for the measurement of pressure and/or air flow. In a preferred embodiment said means is a pressure and/or air flow sensor. Pressure and/or air flow sensors provide information about the amount of air that is passing through the venting lines and furthermore provide information of the state of each cushion. In a preferred embodiment pressure sensors in cushions of the second type are used to detect movements and/or movement intentions of the patient automatically. In another preferred embodiment pressure strain gauges implemented into cushions of the first type and/or into the bed of the imaging device comprising the fixation device are used to monitor the position and/or the movement of the patient. This provides for the advantage that movement or intention of the movement of the patient can be automatically registered and, for example, the fixation device may release the patient to carry out his or her movement and/or the imaging device may not carry out a measurement if a movement is detected.

In another embodiment the fixation device comprises at least one means for the control of the inflation and/or deflation of the cushions. In a preferred embodiment said means is an electronic control system that operates the fixation procedure and processes the data of the means for the measurement of pressure and/or air flow.

In another embodiment of the invention the fixation device is connectable to further control units such as operator panels, computers or remote buttons for the patient.

In another embodiment of the invention the fixation device further comprises at least one mechanical means that support the fixation of the arrangement of the cushions.

In a further embodiment of the invention the fixation device comprises a control means operable by the patient that releases the fixation. Such control means may open the valve(s) and/or control the vacuum and/or air pumps to inflate the cushions of the first type and/or deflate the cushions of the second type. This has the advantage that the patient can easily and quickly release the fixation if, for example, he or she feels uncomfortable. In a preferred embodiment said control means is a button and/or remote button.

In another embodiment the fixation device further comprises a localization system that registers the position and/or the orientation of the fixated body part, preferably the head, of the patient. This has the advantage that these pieces of information may be helpful for the consequent image reconstructions.

In a further embodiment the fixation device of the invention is used for supporting and/or fixating an infant and/or a neonate. This embodiment is particularly advantageous if additional pressure sensors in cushions of the second type are used to detect movements and/or movement intentions of the infant and/or neonate automatically.

Even more preferably, the fixation device additionally comprises a warming system, for example, to keep the patient, infant and/or neonate warm during the examination.

In a further embodiment of the invention the fixation device of the invention is used to support and/or fixate patients suffering from spine kyphosis and/or lordosis, who often find it painful to lie in, for example, an MIT or CT.

In another embodiment the fixation device of the invention is used to fixate a joint of a patient and at the same time apply mechanical stress to said joint by means of at least one of the cushions according to the invention. This has the advantage that additional and valuable diagnostic image information of the joint under stress can be derived from the examination. In a preferred embodiment of the invention, the joint under mechanical stress is a knee, an elbow, an ankle, a wrist, and or a finger.

In another embodiment the fixation device according to the present invention is built into an image device, preferably an MIT, MRI or CT. Thus, the present invention is further directed to any imaging device comprising a fixation device of the present invention. In a further embodiment of the invention the fixation device is provided as a separate appliance. In the latter case, the fixation device may be mounted on a rack.

In a further aspect the invention concerns a method for operating the fixation device or the imaging system according to the invention comprising the step of: a) Deflating the cushions of the first type and inflating the cushions of the second type.

In one embodiment the method further comprises the steps of b) Acquiring image data; and/or c) Inflating the cushions of the first type and deflating the cushions of the second type.

In a preferred embodiment data acquisition is carried out by means of MRI, MIT or CT.

In a preferred embodiment the fixation quality of the fixated body part is measured before the acquisition of the image data in step b). This has the advantage that data acquisition can only be carried out if the fixation of the body part is sufficient to enable a data acquisition of high quality. Thus, high quality image data is only acquired during periods of perfect fixation.

In another preferred embodiment steps a) through c) are carried out at least twice. In other words, the acquisition time of the imaging data is split into at least two periods of equal or different durations. This has the advantage that the patient needs not to be fixated for a longer period of time which he or she may perceive as uncomfortable. Rather, the period of image acquisition is split into two or more periods of shorter duration which will lead to a more pleasant feeling for the patient even during examinations of longer duration.

In a further preferred embodiment the position of the fixated body part is registered for each period of fixation. Thus, if at least two periods of data acquisition are carried out the acquired image data can be processed during the subsequent image reconstructions utilizing this positional information.

In further preferred embodiments the inflation and deflation of the cushions is carried out at the same time or subsequently. Preferably, inflation and deflation are carried out at the same time. If inflation and deflation are carried out subsequently then it is preferred to first deflate the cushion(s) of the first type followed by an inflation of the cushion(s) of the second type.

In another embodiment the method further comprises the step of d) processing the image data. Such a processing of the image data is also known as image reconstruction, wherein the imaging data acquired by them for example, MRI or MIT system are processed and displayed as images. Preferably, at least two sets of imaging data that were acquired during separate periods of fixation are processed together with the data on the position of the fixated body part.

In another preferred embodiment these processed data that can be displayed as images are evaluated by, for example, a physician in a separate step e), in order to derive medical information therefrom. As an example, the images resulting from the image reconstruction may be used by the physician to diagnose an injury of the brain, a fracture of the skull and/or a brain tumor.

### BRIEF DESCRIPTION OF THE DRAWINGS

These and other aspects of the invention will be apparent from and elucidated with reference to the embodiments described hereinafter.

In the drawings:

FIG. 1 shows a preferred embodiment of the fixation device, wherein a head fixation device is built into a CT.

### DETAILED DESCRIPTION OF EMBODIMENTS

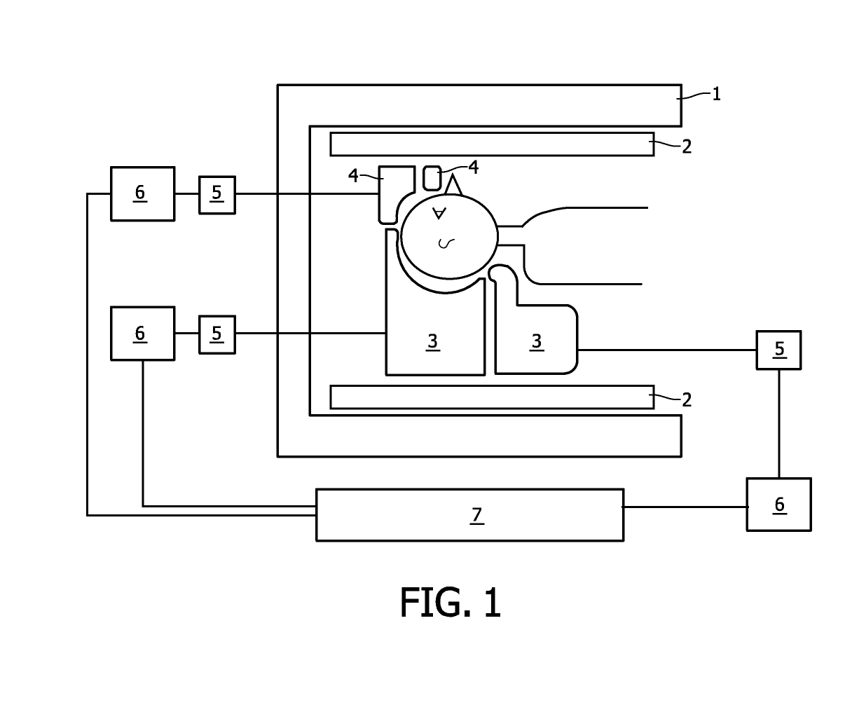

FIG. 1 shows a preferred embodiment of the fixation device according to the invention, wherein the fixation device is a head fixation device built in into an MIT system.

A tomograph 1 is shown, comprising sensors for the image acquisition 2. Furthermore, the tomograph comprises a fixation device according to the present invention comprising four cushions 3, 4.

Cushions 3 are cushions of the first type, while cushions 4 are cushions of the second type. A patient is arranged supine in the tomograph, wherein his lower head and neck rests on the cushions 3 that have taken on the shape of the head and neck, respectively.

The two cushions of the second type 4 are arranged to contact the forehead portion of the head and the area of the root of the nose of the patient, respectively. Thus, no sensitive areas of the head of the patient are contacted.

The casing of the cushions 3, 4 is made from a semi-rigid plastic material and the cushions of the first type 3 comprise Styrofoam, while the cushions of the second type 4 comprise soft plastic foam and contact the object placed on the cushion of the first type.

Cushions 3 and 4 are connected to vacuum pumps/air pumps 6 and valves 5, allowing managing the inflation and deflation of the cushions separately. Furthermore, an electronic control system 7 is shown that is connected to the vacuum pumps/air pumps 6.

Initially, cushions of the first type 3 are filled with air. Hence, they are soft and flexible, and they can easily adapt their shape to the head of the patient. The cushions of the second type 4 are deflated, so that their volume is small. The head can be placed into the image device and movements of the patient are allowed so that the head can be moved into a comfortable position.

When the fixation is activated, the air is removed from cushions 3 by means of the vacuum pumps. These cushions will subsequently keep their shape and become stiff. Thus, the head of the patient is fixed in its position. Furthermore, cushions 4 are inflated at the same time as cushions 3 are deflated which ensures and intensifies the fixation. The process of inflation and deflation is individually controlled for each cushion, utilizing pressure and air flow sensors connected to each cushion.

Furthermore, the fixation quality is measured to decide if MIT imaging should commence. Should the fixation be not strong enough, the previous steps are repeated in order to achieve better fixation.

When an acceptable state of fixation is reached, the position of the head is registered and the gained information is used in subsequent image reconstruction and analysis methods.

Pressure sensors connected to cushions 4 that are inflated at fixation allow the detection of movements and the intentions for movement, respectively. In such cases, the fixation can be released and re-established later on with the head in different position.

The system prevents unacceptable discomfort for the patient by monitoring movements, pressure in the cushions, and duration of the fixation. Additionally, an immediate release of the fixation may also be induced by the patient by pressing a button.

The release from fixation is conducted by opening the valves and filling cushions 3 with air and removing air from cushions 4. This can be done within a few seconds.

The term of image acquisitions is split into several shorter periods of fixation, wherein the release of fixation can be induced by the patient, for example, by intended movements or by pressing a button. Alternatively, the system automatically detects movement and rest detection or defines specific time slices. Images should only be acquired when the head is fixed.

After the data acquisition and the release of the patient the image data are processed by image reconstruction, wherein all sets of imaging data that were acquired during the separate periods of fixation are processed together with the data on the position of the fixated body part.

These processed data are then displayed as images and evaluated by the attending physician, in order to derive medical information therefrom.

While the invention has been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. Fixation device comprising at least two inflatable and deflatable cushions of two different types, wherein
the cushion of the first type (3) is deformable when inflated and not deformable when deflated and
the cushion of the second type (4) is arranged to at least partially expand into the direction of a cushion of the first type upon inflation.

2. The fixation device according to claim 1, wherein a soft material is attached to the surface of the cushion of the second type.

3. The fixation device according to claim 1, wherein the cushion of the second type takes in a predefined shape upon inflation.

4. The fixation device according to claim 1 further comprising at least one vacuum pump and/or air pump (6).

5. The fixation device according to claim 1 further comprising at least one means for the measurement of pressure and/or air flow.

6. The fixation device according to claim 1 further comprising at least one means for the control of the inflation and/or deflation of the cushions (7).

7. The fixation device according to claim 1 further comprising a control means operable by the patient that releases the fixation.

8. The fixation device according to claim 1, wherein the fixation device is a head fixation device.

9. An imaging system (1) comprising the fixation device according to claim 1.

10. A method for operating the fixation device according to claim 1 comprising the step of:
a) Deflating the cushions of the first type (3) and inflating the cushions of the second type (4).

11. The method according to claim 10, further comprising the steps of:
b) Acquiring image data; and/or
c) Inflating the cushions of the first type and deflating the cushions of the second.

12. The method according to claim 11, wherein steps a) through c) are carried out at least twice.

13. The method according to claim 11, wherein the position of the fixated body part is registered for each period of fixation.

14. The method according to claim 11, further comprising the step of processing the image data and deriving medical information therefrom.
